(19) United States
(12) Patent Application Publication
(10) Pub. No.: US 2016/0038033 A1
(43) Pub. Date: Feb. 11, 2016

(54) METHOD, SYSTEM AND APPARATUS FOR QUANTIFYING ORAL HEALTH WITH A TOOTHBRUSH

(71) Applicant: RAKESH M. LAL, CAMBRIDGE, MA (US)

(72) Inventor: RAKESH M. LAL, CAMBRIDGE, MA (US)

(21) Appl. No.: 14/815,141

(22) Filed: Jul. 31, 2015

Related U.S. Application Data
(60) Provisional application No. 61/999,744, filed on Aug. 5, 2014.

(51) Int. Cl. A61B 5/00; A61B 1/04; A61C 17/00; A61B 1/06; A61B 1/07; A61B 1/24; A61B 1/00

(52) U.S. Cl. CPC A61B 5/0088; A61B 5/6887; A61B 5/0013; A61B 5/742; A61B 5/4547; A61B 5/4552; A61B 1/24; A61B 1/04; A61B 1/00009; A61B 5/0017; A61B 1/0646; A61B 1/0684; A61B 1/07; A61B 1/0002; A61C 17/00

(57) ABSTRACT

Systems, methods and apparatus are provided through which in one aspect, a toothbrush system for imaging a dental structure includes a handle, a brush head, a tracking system, an optical system operable to acquire a set of optical absorption samples associated with a set of optical properties and a time, and a set of machine readable instructions operable to receive the three dimensional position of the brush head, receive the optical absorption samples, associate each optical absorption sample with a point on the dental structure, and determine oral health measures associated with each point on the dental structure. In another aspect, a method for quantifying oral health includes acquiring a set of optical absorption samples wherein each sample is associated with a three dimensional point on a dental structure, and computing a set of oral health measures based on the set of optical absorption samples.

### RELATED APPLICATION

This application claims the benefit of U.S. Provisional Application Ser. No. 61/999,744, tiled Aug. 5, 2014 under 35 U.S.C. 119(e).

### FIELD OF THE INVENTION

This invention relates generally to quantifying a subject's oral health with a toothbrush, and more particularly to tracking the three dimensional position of a toothbrush and associating the three dimensional position relative to the user's dentition with spectroscopic signals acquired by optical sensors embedded within the same toothbrush.

### BACKGROUND OF THE INVENTION

Most toothbrushes on the market today are simple devices that comprise a brush head and a handle, with no electronic sensing technology built in to them. Though they serve the primary function of cleaning a person's dental tissue (teeth, gums, etc.), there is a great deal of potential to understanding one's overall oral health by leveraging the, simple toothbrush to capture various optical absorption properties and signals during the course of brushing, and using these optical properties and signals to derive oral health measures. Effectively utilizing these properties and signals captured during the course of brushing to derive oral health measures however usually requires information related to the segment, component, part and type of the dental tissue from which the signal was acquired. As such, tracking the position of the toothbrush relative to a user's dentition during the process of brushing one's teeth is critical to discerning trends in an individual's oral health.

Some more advanced toothbrush systems employ tracking of brush strokes via simple sensors such as accelerometers or gyroscopes embedded within the brush. Other technologies such as position sensing may be capable of tracking the three dimensional position of a toothbrush relative to a stationary reference, such as the toothbrush's charging base. The limitation of these approaches is that they do not track the position relative to the user's dentition, and thus any motion of the teeth, jaw, head, or overall body may result in spurious data.

Other methods of monitoring oral health may include more sophisticated devices such as X-ray scanners, CT scanners, optical imaging scanners, or other imaging devices typically available at a dentist's office, The limitation of these devices is that their use is restricted to a patient's visit to a dentist, thus reducing the number of time points that can be measure. This in turn reduces the ability to discern any oral health trends associated with an individual.

For the reasons stated above, and for other reasons stated below which will become apparent to those skilled in the art upon reading and understanding the present specification, there is a need in the art for a toothbrush that is capable of being tracked relative to a user's dentition, during the process of brushing. There is also a need for acquiring optical absorption properties and signals during the process of brushing and as they relate to a person's dentition, and using these optical signals to derive oral health measures.

### BRIEF DESCRIPTION OF THE INVENTION

The above-mentioned shortcomings, disadvantages and problems are addressed herein, which will be understood by reading and studying the following specification.

In one aspect, a toothbrush system for imaging a dental structure includes a handle, a brush head having a three dimensional position, a tracking system operable to determine the three dimensional position of the brush head relative to the dental structure, an optical system operable to acquire a set of optical absorption samples wherein each optical absorption sample is associated with a set of optical properties and a time, and a first set of machine readable instructions embodied in a non-transitory computer readable storage medium, operable to receive the three dimensional position of the brush head, further operable to receive the set of optical absorption samples, further operable to associate each optical absorption sample with a three dimensional point on the dental structure, and further operable to determine a set of oral health measures associated with each three dimensional point on the dental structure based on the sets of optical properties.

In another aspect, a method for quantifying oral health includes acquiring a set of optical absorption samples wherein each sample is associated with a three dimensional point on a dental structure, and computing a set of oral health measures based on the set of optical absorption samples.

Apparatus, systems, and methods of varying scope are described herein. In addition to the aspects and advantages described in this summary, further aspects and advantages will become apparent by reference to the drawings and by reading the detailed description that follows.

### BRIEF DESCRIPTION OF THE DRAWINGS

FIG. 1 is a block diagram illustrating a system of quantifying an individual's oral health using a toothbrush.

FIG. 2 is a cross section block diagram of an apparatus according to an aspect to compute oral health measures based on acquiring optical absorption properties associated with a dental structure.

FIG. 3 is a cross section block diagram of an apparatus according to an aspect to compute oral health measures based on acquiring optical absorption and polarization properties associated with a dental structure.

FIG. 4 is a flowchart of a method to compute oral health measures based on a set of optical absorption samples.

FIG. 5 is a flowchart of a method to acquire an optical absorption sample, which is associated with a three dimensional point on a dental structure.

### DETAILED DESCRIPTION OF THE INVENTION

In the following detailed description, reference is made to the accompanying drawings that form a part hereof, and in which is shown by way of illustration specific aspects which may be practiced. These aspects are described in sufficient detail to enable those skilled in the art to practice the aspects, and it is to be understood that other aspects may be utilized and that logical, mechanical, electrical and other changes may be made without departing from the scope of the aspects. The following detailed description is, therefore, not to be taken in a limiting sense.

The detailed description is divided into four sections. In the first section, a system level overview is described. In the second section, apparatus of aspects are described. In the third section, aspects of methods are described. Finally, in the fourth section, a conclusion of the detailed description is provided.

### System Level Overview

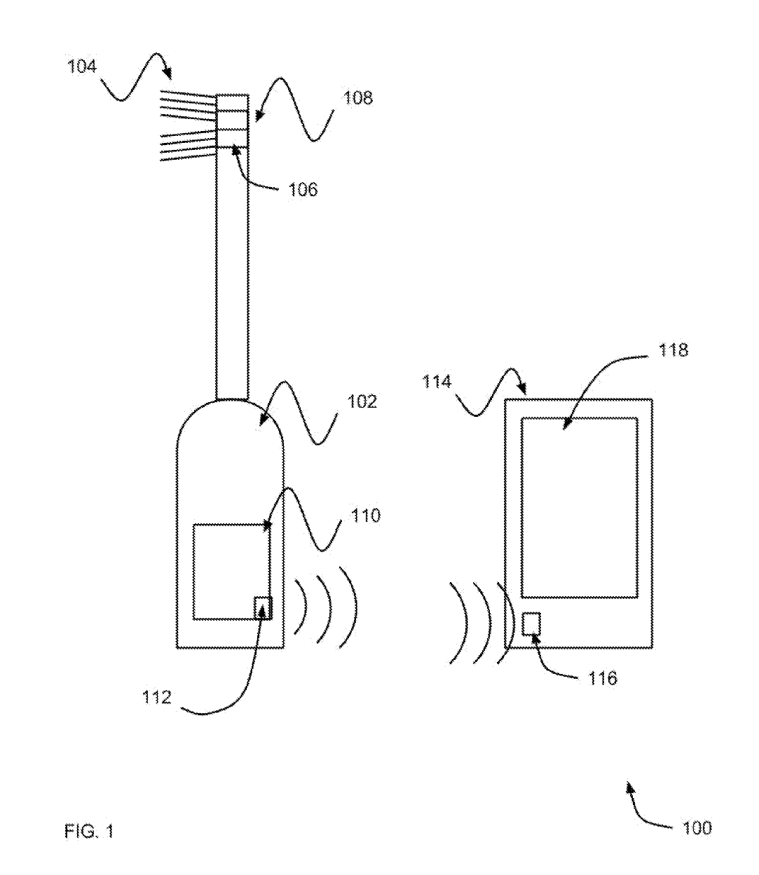

FIG. 1 is a block diagram illustrating a system of quantifying an individual's oral health using a toothbrush. System 100 solves the need in the art to accurately track the three dimensional position of a toothbrush relative to a user's dental structure, and to acquire key optical signal data that can be used to derive oral health measures.

System 100 includes a handle 102, a brush head 104 having a three dimensional position, a tracking system 106 and operable to determine the three dimensional position of the brush head relative to the dental structure, an optical system 108 operable to acquire a set of optical absorption samples wherein each optical sample is associated with a set of optical properties and a time, and a set of machine readable instructions embodied in a non-transitory computer readable storage medium 110, operable to receive the set of set of optical absorption samples, further operable to associate each absorption sample with a three dimensional point on the dental structure, and further operable to determine a set of oral health measures associated with each three dimensional point on the dental structure based on the set of optical properties. In some aspects, System 100 further includes a communication system 112 operable to transmit the set of optical absorption samples and the associated three dimensional points, and a visualization system 114 operable to receive the set of oral health measures and the associated three dimensional points, and further operable to render a representation of the set of oral health measures and the associated three dimensional points. In other aspects, the visualization system further includes a receiver 116 operable to receive the set of oral health measures and the associated three dimensional points on the dental structure being imaged, and a display system 118 operable to render the set of oral health measures and the associated three dimensional points.

Component 106 solves the need in the art to track the three dimensional position of the brush head over the course of time, relative to the dental structure being imaged.

Component 108 solves the need in the art to acquire a set optical absorption samples. Each optical sample is further associated with a set of optical properties and a time.

Component 110 solves the need in the art to associate a three dimensional point on the dental structure being imaged with each optical absorption sample. Component 110 further solves the need in the art to determine a set of oral health measures based on the set of optical absorption samples, the set of optical properties associated with each optical absorption sample, and the three dimensional point on the dental structure associated with each optical absorption sample. In some aspects, the oral health measures include a set of tooth decay measures, a set of gingival recession measures, a set of periodontal health measures, a set of enamel thickness measures, and a set of plaque extent measures.

The system level overview of the operation of an aspect is described in this section of the detailed description. The tracking system determines the three dimensional position of the brush head relative to the dental structure being imaged. In some aspects, the tracking system further includes a light source, a camera operable to capture a set of video frames, and a second set of machine readable instructions embodied in a non-transitory computer readable storage medium, operable to receive the set of video frames, and further operable to determine a three dimensional position of the brush head relative to the dental structure associated with each video flame in the set of video frames. In other aspects, the light source further includes a light source operable to protect a pattern of light onto the dental structure being imaged. In other aspects, the camera system further includes a light field camera operable to capture a set of video frames, wherein each video frame is a light field image. In another aspect the light field image includes an image intensity function that varies with spatial co-ordinates as well as angular co-ordinates. In one aspect, mathematical constructs exist to convert a light field image to a set of standard images each of which include an intensity function that varies with spatial co-ordinates only, wherein each image focused at a different focal depth.

In some aspect, the tracking system includes a stationary base operable to transmit a signal, a set of three or more receivers embedded within the toothbrush system operable to receive the signal, and a second set of machine readable instructions embodied in a non-transitory computer readable storage medium, operable to determine a three dimensional position of the brush head relative to the dental structure based on the received signal. In some aspects, the second set of machine instructions further includes a set of machine readable instructions embodied in a non-transitory computer readable storage medium, operable to determine a three dimensional position of the brush head relative to the dental structure based on the attenuation of the received signal experienced at each of the receivers in the set of three or more receivers. In other aspects, the second set of machine instructions further includes a set of machine readable instructions embodied in a non-transitory computer readable storage medium, operable to determine a three dimensional position of the brush head relative to the dental structure based on the time shift of the received signal experienced at each of the receivers in the set of three or more receivers. In yet another aspect, the signal transmitted by the stationary base includes a radiofrequency signal. In other aspects, the signal transmitted by the stationary base includes an infrared signal. In yet another aspect, the signal transmitted by the stationary base includes an acoustic signal.

While the system 100 is not limited to any particular tracking system, optical system and set of machine readable instructions, fm sake of clarity a simplified tracking system, optical system and machine readable instructions are described.

### Apparatus Aspects

In the previous section, a system level overview of the operation of an aspect was described. In this section, the particular apparatus of such an aspect are described by reference to a series of diagrams.

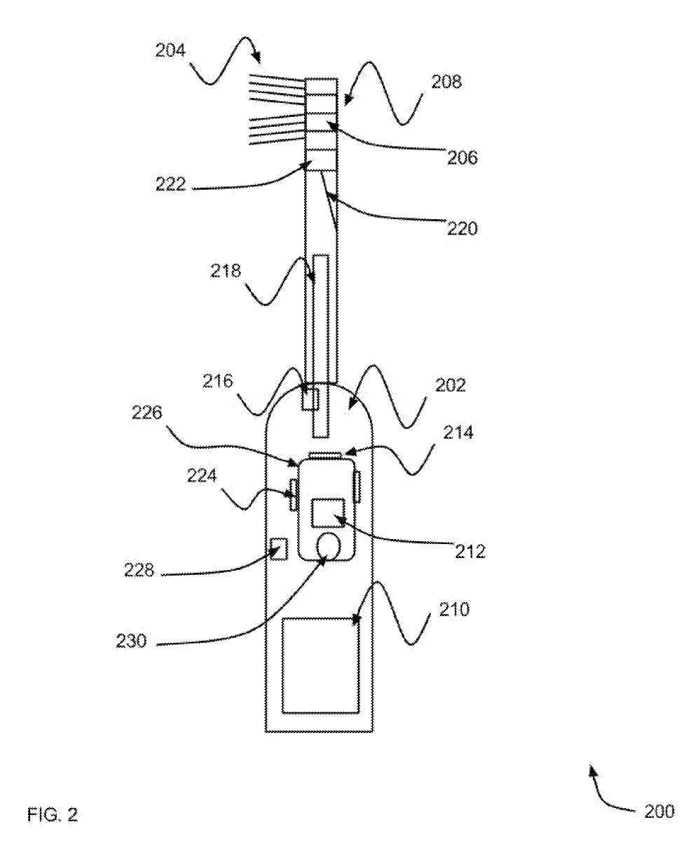

FIG. 2 is a cross section block diagram of apparatus 200 according to an aspect to compute oral health measures based on acquiring optical absorption properties associated with a dental structure. Apparatus 200 solves the need in the art to acquire optical absorption properties associated with dental structures.

Apparatus 200 includes a handle 202, a brush head 204 having a three dimensional position, a tracking system 206 operable to determine the three dimensional position of the brush head relative to the dental structure, a light source 208 operable to illuminate the dental structure being imaged, and a set of machine readable instructions embodied in a non-transitory computer readable storage medium 210, operable to receive a set of set of optical absorption samples, further operable to associate each absorption sample with a three dimensional point on the dental structure, and further operable to determine a set of oral health measures associated with each three dimensional point on the dental structure based on the set of optical properties. In some aspects, apparatus 200 further includes a light source 212 operable to emit a broadband beam of light, an optical filtering system 214 operable to filter the broadband beam of light into a filtered beam having a set of optical properties that vary over time, a first photodiode 216 operable to receive a fixed subset of the filtered beam and generate a first current signal proportional to the intensity of the received subset of the filtered beam, an optical delivery system 218 operable to deliver a fixed subset of the filtered beam to the first photodiode and the remaining subset of the filtered beam to the dental structure, and a second photodiode 222 operable to receive a light signal reflected from the dental structure and generate a second current signal proportional to the intensity of the reflected light signal. In some aspects the optical delivery system further includes a reflection element 220 operable to reflect the remaining subset of the filtered beam onto the dental structure.

In some aspects, the optical filtering system of apparatus 200 further includes a set of optical filter elements 224 each operable to filter the broadband beam of light into a filtered beam having a set of optical properties, mounted on a transport system 226 wherein each optical filter element is abutted by an opaque segment on either side, and wherein the broadband beam of light is occluded by either one optical filter element or opaque segment at any time, a motor 230 operable to rotate the transport system, and an encoder 228 operable to determine which optical filter element or opaque segment is occluding the broadband beam of light at any time.

In other aspects, the optical filtering system further includes a set of machine readable instructions embodied in a non-transitory computer readable storage medium, operable to derive each optical absorption sample from the first current signal at a time when the broadband beam is occluded by a filter element, the first current signal at a time when the broadband beam is occluded by an opaque segment, the second current signal at a time when the broadband beam is occluded by the same filter element, and the second current at a time when the broadband beam is occluded by the same opaque segment.

In yet other aspects, the set of optical properties further includes a set of wavelength values.

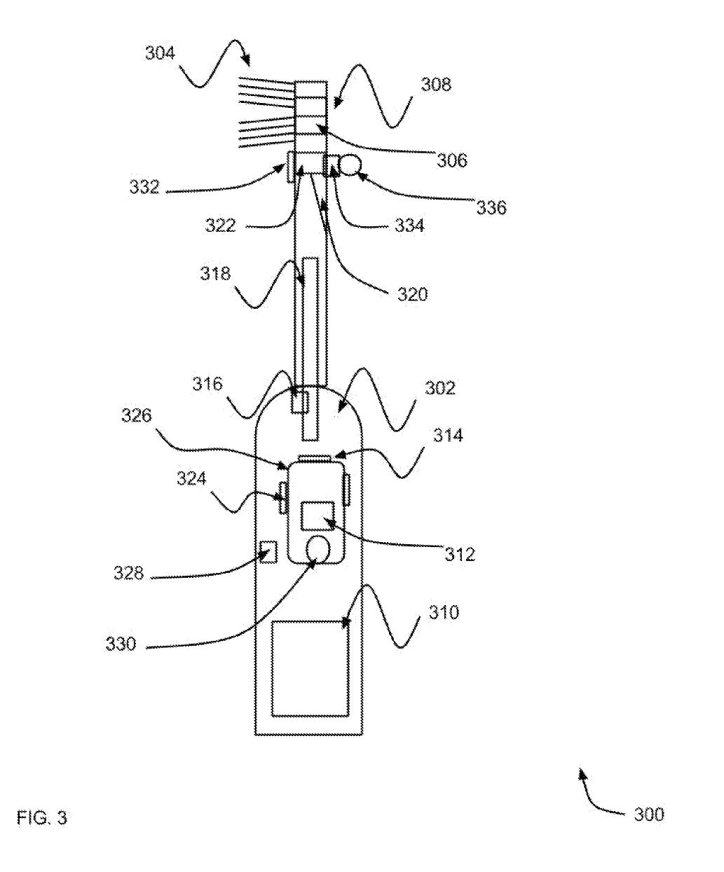

FIG. 3 is a cross section block diagram of apparatus 300 according to an aspect to compute oral health measures based on acquiring optical absorption and polarization properties associated with a dental structure. Apparatus 300 solves the need in the art to acquire optical absorption and polarization properties associated with dental structures.

Apparatus 300 includes a handle 302, a brush head 304 having a three dimensional position, a tracking system 306 operable to determine the three dimensional position of the brush head relative to the dental structure, a light source 308 operable to illuminate the dental structure being imaged, and a set of machine readable instructions embodied in a non-transitory computer readable storage medium 310, operable to receive a set of set of optical absorption samples, further operable to associate each absorption sample with a three dimensional point on the dental structure, and further operable to determine a set of oral health measures associated with each three dimensional point on the dental structure based on the set of optical properties. In some aspects, apparatus 300 further includes a light source 312 operable to emit a broadband beam of light, a first optical filtering system 314 operable to filter the broadband beam of light into a filtered beam having a set of optical properties that vary over time, a first photodiode 316 operable to receive a fixed subset of the filtered beam and generate a first current signal proportional to the intensity of the received subset of the filtered beam, an optical delivery system 318 operable to deliver a fixed subset of the filtered beam to the first photodiode and the remaining subset of the filtered beam to the dental structure, a second optical filtering system operable to receive a light signal reflected from the dental structure and further operable to filter the received light signal into a filtered reflected signal, and a second photodiode 322 operable to receive a light signal reflected from the dental structure and generate a second current signal proportional to the intensity of the reflected light signal. In some aspects the optical delivery system further includes a reflection element 320 operable to reflect the remaining subset of the filtered beam onto the dental structure.

In some aspects, the first optical filtering system further includes a set of optical filter elements 324 each operable to filter the broadband beam of light into a filtered beam having a set of optical properties, mounted on a first transport system 326 wherein each optical filter element is abutted by an opaque segment on one side and another optical filter element on the other side, and wherein two abutting optical filter elements are each operable to filter the broadband beam into a filtered beam having the same set of optical properties except that one filtered beam is polarized while the other is not polarized, and wherein the broadband beam of light is occluded by either one optical filter element or opaque segment at any time, a motor 330 operable to rotate the first transport system at a fixed speed, and an encoder 328 operable to determine which optical filter element or opaque segment is occluding the broadband beam of light at an time.

In other aspects, the second optical filtering system further includes a set of optical polarization elements 332 each operable to polarize the received light signal into a polarized beam, mounted on a second transport system wherein each optical polarization element is abutted by a pair of transparent segment on either side, and wherein the received light signal is occluded by either one optical polarization element or transparent segment at any time, a motor 336 operable to rotate the second transport system at a fixed speed wherein the rotation of the second transport system is synchronized with the rotation of the first transport system, and an encoder 334 operable to determine which optical polarization element or transparent segment is occluding the received light signal at any time.

In yet other aspects, the optical system includes a set of machine readable instructions embodied in a non-transitory computer readable storage medium, operable to derive each optical absorption sample from the first current signal at a time when the broadband beam is occluded by a filter element, the first current signal at a time when the broadband beam is occluded by filter element with polarized filtered beam, the first current signal at a time when the broadband beam is occluded by an opaque segment, the second current signal at a time when the broadband beam is occluded by the same filter element and the received light signal is occluded by a transparent segment, the second current signal at a time when the broadband beam is occluded by the same filter element with polarized filtered beam and the received light signal is occluded by a polarization element, and the second current at a time when the broadband beam is occluded by the same opaque segment and the received light signal is occluded by a transparent segment.

### Method Aspects

In the previous section, apparatus of the operation of an aspect was described. In this section, the particular methods performed by usage of such an aspect are described by reference to a series of flowcharts.

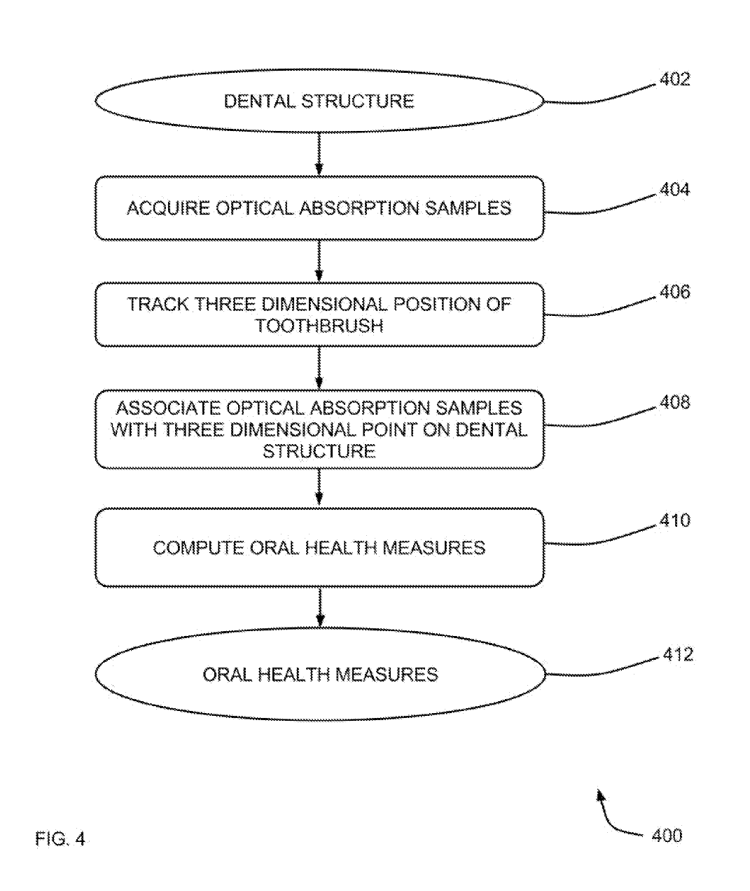

FIG. 4 is a flowchart of method 400 to compute oral health measures based on a set of optical absorption samples. Method 400 solves the need in the art to compute a set of oral health measures associated with three dimensional points on a dental structure

Method 400 includes a dental structure 402 being imaged, acquiring a set of optical absorption samples 404, wherein each sample is associated with a three dimensional point on a dental structure, and computing a set of oral health measures 410 based on the set of optical absorption samples, resulting in a set of oral health measures 412. In some aspects, method 400 further includes tracking a three dimensional position of a toothbrush 406 relative to the dental structure, and associating the each sample in the set of optical absorption samples with the three dimensional point on the dental structure 408 based on the tracked three dimensional position of the toothbrush.

In some aspects, each sample in the set of optical absorption samples is associated with a set of optical properties. In another aspect, the set of optical properties includes a wavelength. In other aspects, the set of optical properties includes a polarization state.

In some aspects, the set of oral health measures further comprises a tooth decay measure, a gingival recession measure, a periodontal health measure, an enamel thickness measure, and a plaque extent measure.

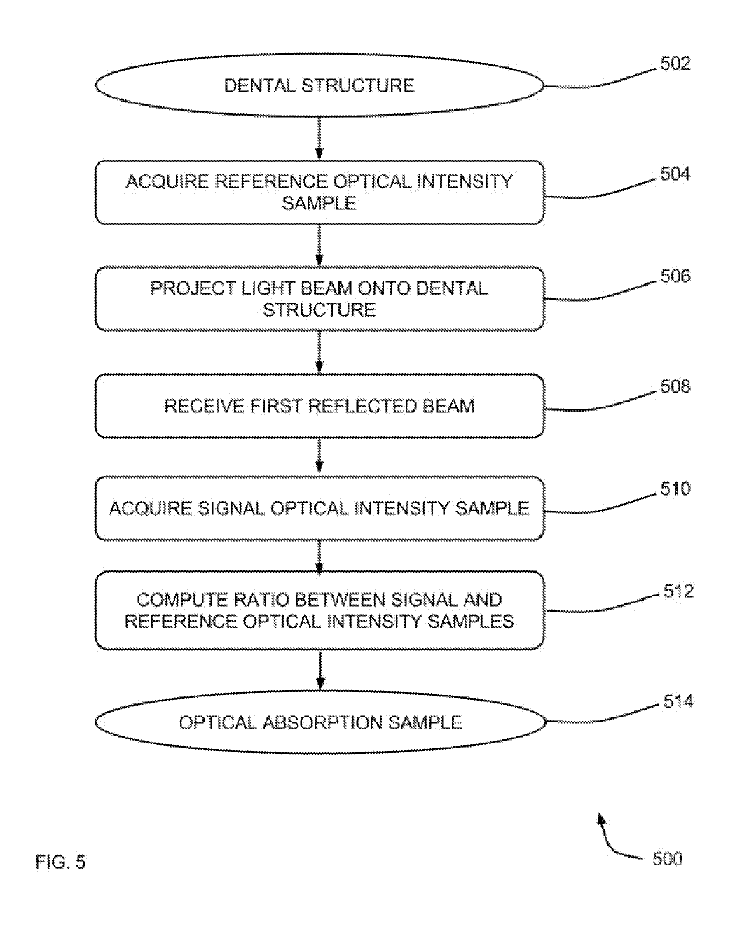

FIG. 5 is a flowchart of method 500 to acquire an optical absorption sample, which is associated with a three dimensional point on a dental structure. Method 500 solves the need in the art to acquire an optical absorption sample, which is associated with a three dimensional point on a dental structure.

Method 500 includes a dental structure 502 being imaged, acquiring a reference optical intensity sample 504 associated with a beam of light having a particular wavelength, projecting the beam of light onto the dental structure 506, receiving a first reflected beam 508 of light reflected by the dental structure, acquiring a signal optical intensity sample 510 based on the first reflected beam, and computing a ratio between the signal and reference optical intensity samples 512, to yield an optical absorption sample 514.

In some aspects, method 500 further includes acquiring an ambient signal optical intensity sample, and subtracting the ambient signal optical intensity sample from the signal optical intensity sample. In other aspects, method 500 further includes acquiring a surface reflection optical intensity sample, and subtracting the surface reflection optical intensity sample from the signal optical intensity sample.

In some aspects, acquiring the surface reflection optical intensity sample further includes acquiring a reference polarized optical intensity sample associated with a polarized beam of light having a particular wavelength, projecting the polarized beam of light onto the dental structure, receiving a second reflected beam of light reflected by the dental structure, passing the second reflected beam through a polarizing filter to generate a polarized reflected beam, acquiring a signal polarized optical intensity sample based on the polarized reflected beam, and scaling the signal polarized optical intensity sample by a ratio between the reference optical intensity sample and the reference polarized optical intensity sample.

### Conclusion

A system, apparatus and method for quantifying oral health with a toothbrush is described. Although specific aspects are illustrated and described herein, it will be appreciated by those of ordinary skill in the art that any arrangement which is calculated to achieve the same purpose may be substituted for the specific aspects shown. This application is intended to cover any adaptations or variations. For example, although described in terms of an optical system comprising a light source, an optical filtering system, a first photodiode, an optical delivery system, and a second photodiode, one of ordinary skill in the art will appreciate that implementations can be made using any other system capable of constructing an optical absorption map of a dental structure, which can be used to derive oral health measures. Additionally, although described in terms of oral health measures such as enamel thickness, gingival recession, tooth decay, plaque extent and periodontal health, one of ordinary skill in the art will appreciate that implementations can also be made that reflect any other oral health metric of interest that can be acquired or derived from optical absorption properties of dental structures.

In particular, one of skill in the art will readily appreciate that the names of the methods and apparatus are not intended to limit aspects. Furthermore, additional methods and apparatus can be added to the components, functions can be rearranged among the components, and new components to correspond to future enhancements and physical devices used in aspects can be introduced without departing from the scope of aspects. One of skill in the art will readily recognize that aspects are applicable to future physiological and oral health measures that can be acquired, derived or interpreted, different methods of acquiring three dimensional position tracking data, and new algorithms to compute oral and physiological health measures from optical absorption data.

The terminology used in this application is meant to include all systems to quantify oral health using a toothbrush, and alternate technologies which provide the same functionality as described herein.

What is claimed:

1. A toothbrush system for imaging a dental structure comprising:
a handle;
a brush head having a three dimensional position;
a tracking system operable to determine the three dimensional position of the brush head relative to the dental structure;
an optical system operable to acquire a set of optical absorption samples wherein each optical absorption sample is associated with a set of optical properties and a time; and
a first set of machine readable instructions embodied in a non-transitory computer readable storage medium, operable to receive the three dimensional position of the brush head, further operable to receive the set of optical absorption samples, further operable to associate each optical absorption sample with a three dimensional point on the dental structure, and further operable to determine a set of oral health measures associated with each three dimensional point on the dental structure based on the sets of optical properties.

2. The toothbrush system of claim 1 further comprising:
a communication system operable to transmit the set of optical absorption samples and the associated three dimensional points; and
a visualization system operable to receive the set of oral health measures and the associated three dimensional points, and further operable to render a representation of the set of absorption samples and the associated three dimensional points.

3. The toothbrush system of claim 1 wherein the set of oral health measures further comprises the set of a tooth decay measure, a gingival recession measure, a periodontal health measure, an enamel thickness measure, and a plaque extent measure.

4. The toothbrush system of claim 1 wherein the tracking system further comprises:
a light source;
a camera operable to capture a set of video frames; and
a second set of machine readable instructions embodied in a non-transitory computer readable storage medium, operable to receive the set of video frames, and further operable to determine a three dimensional position of the brush head relative to the dental structure associated with each video frame in the set of video frames.

5. The toothbrush system of claim 4 wherein the light source further comprises a light source operable to project a pattern of light onto the dental structure.

6. The toothbrush system of claim 4 wherein the camera further comprises a light field camera operable to capture a set of video frames, wherein each video frame is a light field image.

7. The toothbrush system of claim 1 wherein the tracking system further comprises:
a stationary base operable to transmit a signal;
a set of three or more receivers embedded within the toothbrush system operable to receive the signal; and
a second set of machine readable instructions embodied in a non-transitory computer readable storage medium, operable to determine a three dimensional position of the brush head relative to the dental structure based on the received signal.

8. The toothbrush system of claim 7 wherein the second set of machine instructions further comprises a set of machine readable instructions embodied in a non-transitory computer readable storage medium, operable to determine a three dimensional position of the brush head relative to the dental structure based on the attenuation of the received signal experienced at each of the receivers in the set of three or more receivers.

9. The toothbrush system of claim 7 wherein the second set of machine instructions further comprises a set of machine readable instructions embodied in a non-transitory computer readable storage medium, operable to determine a three dimensional position of the brush head relative to the dental structure based on the time shift of the received signal experienced at each of the receivers in the set of three or more receivers.

10. The toothbrush system of claim 7 wherein the signal further comprises one from the set of a radiofrequency signal, an infrared signal, and an acoustic signal.

11. The toothbrush system of claim 1 wherein the optical system further comprises:
a light source operable to emit a broadband beam of light;
an optical filtering system operable to filter the broadband beam of light into a filtered beam having a set of optical properties that vary over time;
a first photodiode operable to receive a fixed subset of the filtered beam and generate a first current signal proportional to the intensity of the received subset of the filtered beam;
an optical delivery system operable to deliver a fixed subset of the filtered beam to the first photodiode and the remaining subset of the filtered beam to the dental structure; and
a second photodiode operable to receive a light signal reflected from the dental structure and generate a second current signal proportional to the intensity of the reflected light signal.

12. The toothbrush system of claim 11 wherein the optical filtering system further comprises:
a set of optical filter elements each operable to filter the broadband beam of light into a filtered beam having a set of optical properties, mounted on a transport system wherein each optical filter element is abutted by an opaque segment on either side, and wherein the broadband beam of light is occluded by either one optical filter element or opaque segment at any time;
a motor operable to rotate the transport system; and
an encoder operable to determine which optical filter element or opaque segment is occluding the broadband beam of light at any time.

13. The toothbrush system of claim 12 wherein the optical filtering system further comprises:
a set of machine readable instructions embodied in a non-transitory computer readable storage medium, operable to derive each optical absorption sample from the first current signal at a time when the broadband beam is occluded by a filter element, the first current signal at a time when the broadband beam is occluded by an opaque segment the second current signal at a time when the broadband beam is occluded by the same filter element, and the second current at a time when the broadband beam is occluded by the same opaque segment.

14. The toothbrush system of claim 13 wherein the set of optical properties further comprises a wavelength value.

15. The toothbrush system of claim 1 wherein the optical system further comprises:
a light source operable to emit a broadband beam of light;
a first optical filtering system operable to filter the broadband beam of light into a filtered beam having a set of optical properties that vary over time;
a first photodiode operable to receive a fixed subset of the filtered beam and generate a first current signal proportional to the intensity of the received subset of the filtered beam;
an optical delivery system operable to deliver a fixed subset of the filtered beam to the first photodiode and the remaining subset of the filtered beam to the dental structure;
as second optical filtering system operable to receive a light signal reflected from the dental structure and further operable to filter the received light signal into a filtered reflected signal; and
a second photodiode operable to receive the filtered reflected signal and generate a second current signal proportional to the intensity of the filtered reflected signal.

16. The toothbrush system of claim 15 wherein the first optical filtering system further comprises:
a set of optical filter elements each operable to filter the broadband beam of light into a filtered beam having a set of optical properties, mounted on a first transport system wherein each optical filter element is abutted by an opaque segment on one side and another optical filter element on the other side, and wherein two abutting optical filter elements are each operable to filter the broadband beam into a filtered beam having the same set of optical properties except that one filtered beam is polarized while the other is not polarized, and wherein the broadband beam of light is occluded by either one optical filter element or opaque segment at any time;
a motor operable to rotate the first transport system at a fixed speed; and
an encoder operable to determine which optical filter element or opaque segment is occluding the broadband beam of light at any time.

17. The toothbrush system of claim 16 wherein the second optical filtering system further comprises:
a set of optical polarization elements each operable to polarize the received light signal into a polarized beam, mounted on a second transport system wherein each optical polarization element is abutted by a pair of transparent segment on either side, and wherein the received light signal is occluded by either one optical polarization element or transparent segment at any time;
a motor operable to rotate the second transport system at a fixed speed wherein the rotation of the second transport system is synchronized with the rotation of the first transport system; and
an encoder operable to determine which optical polarization element or transparent segment is occluding the received light signal at any time.

18. The toothbrush system of claim 17 wherein the optical system further comprises:
a set of machine readable instructions embodied in a non-transitory computer readable storage medium, operable to derive each optical absorption sample from the first current signal at a time when the broadband beam is occluded by a filter element, the first current signal at a time when the broadband beam is occluded by filter element with polarized filtered beam, the first current signal at a time when the broadband beam is occluded by an opaque segment, the second current signal at a time when the broadband beam is occluded by the same filter element and the received light signal is occluded by a transparent segment, the second current signal at a time when the broadband beam is occluded by the same filter element with polarized filtered beam and the received light signal is occluded by a polarization element, and the second current at a time when the broadband beam is occluded by the same opaque segment and the received light signal is occluded by a transparent segment.

19. The toothbrush system of claim 18 wherein the set of optical properties further comprises a wavelength value.

20. A method for quantifying oral health comprising:
acquiring a set of optical absorption samples wherein each sample is associated with a three dimensional point on a dental structure; and
computing a set of oral health measures based on the set of optical absorption samples.

21. The method of claim 20 further comprising:
tracking a three dimensional position of a toothbrush relative to the dental structure; and
associating the each sample in the set of optical absorption samples with the three dimensional point on the dental structure based on the tracked three dimensional position of the toothbrush.

22. The method of claim 21 wherein each sample in the set of optical absorption samples is associated with a set of optical properties.

23. The method of claim 22 wherein the set of optical properties further comprises:
a wavelength; and
a polarization state.

24. The method of claim 23 wherein acquiring each sample in the set of optical absorption samples further comprises:
acquiring a reference optical intensity sample associated with a beam of light having a particular wavelength;
projecting the beam of light onto the dental structure;
receiving a first reflected beam of light reflected by the dental structure;
acquiring a signal optical intensity sample based on the first reflected beam; and
computing a ratio between the signal and reference optical intensity samples.

25. The method of claim 24 further comprising:
acquiring an ambient signal optical intensity sample; and
subtracting the ambient signal optical intensity sample from the signal optical intensity sample.

26. The method of claim 25 further comprising:
acquiring a surface reflection optical intensity sample; and
subtracting the surface reflection optical intensity sample from the signal optical intensity sample.

27. The method of claim 26 wherein acquiring the surface reflection optical intensity sample further comprises:
acquiring a reference polarized optical intensity sample associated with a polarized beam of light having a particular wavelength;
projecting the polarized beam of light onto the dental structure;
receiving a second reflected beam of light reflected by the dental structure;
passing the second reflected beam through a polarizing filter to generate a polarized reflected beam;
acquiring a signal polarized optical intensity sample based on the polarized reflected beam; and
scaling the signal polarized optical intensity sample by a ratio between the reference optical intensity sample and the reference polarized optical intensity sample.

28. The toothbrush system of claim 20 wherein the set of mal health measures further comprises a tooth decay measure, a gingival recession measure, a periodontal health measure, an enamel thickness measure, and a plaque extent measure.
